(19) United States
(12) Patent Application Publication
(10) Pub. No.: US 2007/0281331 A1
(43) Pub. Date: Dec. 6, 2007

(54) METHOD OF TRACKING SWIMMING PATH OF BACTERIUM

(76) Inventors: Sangmo KOO, Gyeonggi-do (KR); Songwan JIN, Seoul (KR); Yongku LEE, Gyeonggi-do (KR); Jung Yul YOO, Seoul (KR); Jae-young SUNG, Seoul (KR); Chang-Soo LEE, Daejeon (KR)

Correspondence Address: LADAS & PARRY LLP, 224 SOUTH MICHIGAN AVENUE, SUITE 1600, CHICAGO, IL, 60604, US

(21) Appl. No.: 11/624,380

(22) Filed: Jan. 18, 2007

(30) Foreign Application Priority Data
Jun. 6, 2006 (KR) 10-2006-0050620

(51) Int. Cl. C12Q 1/04; G06T 7/20

(52) U.S. Cl. 435/34; 382/128

(57) ABSTRACT

Provided is a method of tracking a swimming path of a bacterium which can exactly track the swimming path of the bacterium by modeling the bacterium as ellipsoidal in shape based on an image of the bacterium obtained while the ellipsoidal bacterium swims near a solid surface. The method of tracking a swimming path of a bacterium which is formed in an ellipsoidal shape and swims in a swimming space formed between a solid surface and an imaginary surface parallel to the solid surface includes the steps of: transfecting a fluorescent gene into the bacterium; arranging the bacterium in the swimming space to swim; totally reflecting light irradiated to the solid surface, and forming an evanescent field in the swimming space; taking a picture of the bacterium expressing the fluorescent gene, which emits light in the evanescent field, at respective moments while the bacterium swims, and obtaining an image of the bacterium expressing the fluorescent gene at the respective moments; and fitting the image of the bacterium obtained at the respective moments to an ellipsoidal shape, and setting the shape of the bacterium as the ellipsoidal shape and a position of the bacterium with respect to the solid surface.

### CROSS-REFERENCE TO RELATED APPLICATION

This application claims the benefit of Korean Patent Application No. 10-2006-0050620, filed on Jun. 6, 2006, the disclosure of which is hereby incorporated herein by reference in its entirety.

### BACKGROUND OF THE INVENTION

1. Field of the Invention

The present invention relates to a method of tracking a swimming path of a bacterium, and more particularly, to a method of tracking a swimming path of an ellipsoidal bacterium swimming near a solid surface.

2. Description of the Related Art

It is widely known that while a bacterium exhibits random movement in an open space, it moves with a certain orientation, for example, exhibits circular movement, near a solid surface, for example, in a swimming space formed within 10 μm of a solid surface. Recently, various attempts are being made to apply such bacterial movement to industry, for example, bio-filters, bio-pumps, bio-motors, and production of bio-energy.

In order to realize such industrial applications, it is first necessary to precisely track a swimming path of a bacterium. Consequently, research into bacterial swimming path tracking has been steadily progressing. As a result of this research, a method of setting the center of a bacterium using a Gaussian fitting method in order to track its swimming path has been widely applied.

The method of tracking a swimming path of a bacterium using the Gaussian fitting method is as follows. First, a fluorescent gene, for example, a green fluorescent protein (GFP) gene, is transfected into a bacterium, the bacterium in which the GFP gene is expressed emits light, and pictures of the bacterium are taken at different moments while the bacterium swims near a solid surface, so as to obtain two-dimensional images of the bacterium such as that illustrated in the top portion of FIG, 1 at the respective moments. The two dimensional images of the bacterium are fitted by the Gaussian fitting method to model the bacterium as spherical in shape, as marked by a dotted line in FIG. 1, and to set a spot with the highest emission intensity as the center of the bacterium, as illustrated in the bottom portion of FIG. 1. Then, comparing the emission intensity at the center of the bacterium with the rest of the bacterium, the distance between the bacterium's center and the solid surface is set. After setting the center of the bacterium near the solid surface, the centers of the bacterium at the respective moments are connected by a line to obtain the swimming path of the bacterium. Here, the three-dimensional shape of the bacterium is obtained as a sphere having a constant radius from the center of the bacterium set near the solid surface.

As described above, in the conventional method of tracking a swimming path of a bacterium, the shape of the bacterium is set as a sphere regardless of the kind of a bacterium. However, in the case of tracking a swimming path of an ellipsoidal bacterium such as RP437, the shape of the bacterium is also modeled as a sphere even though it is actually ellipsoidal, and thus the actual center of the ellipsoidal bacterium may not be set accurately. Moreover, in the case of the ellipsoidal bacterium, relative angles between the bacterium's major axis and the solid surface, and between its minor axis and the solid surface, differ depending on the position of the bacterium. However, the conventional method cannot set exact relative angles. Thus, the conventional method cannot exactly track the swimming path of the ellipsoidal bacterium.

### SUMMARY OF THE INVENTION

An embodiment of the invention provides a method of tracking a swimming path of a bacterium that can accurately track the bacterial swimming path by modeling a bacterium as an ellipsoid based on an image of the bacterium obtained while the ellipsoidal bacterium swims near a solid surface.

In one aspect, the invention is directed to a method of tracking a swimming path of a bacterium which is formed in an ellipsoidal shape and swims in a swimming space formed between a solid surface and an imaginary surface parallel to the solid surface, the method comprising the steps of: transfecting a fluorescent gene into the bacterium; arranging the bacterium in the swimming space to swim; totally reflecting light irradiated to the solid surface, and forming an evanescent field in the swimming space; taking a picture of the bacterium expressing the fluorescent gene, which emits light in the evanescent field, at respective moments while the bacterium swims, and obtaining an image of the bacterium expressing the fluorescent gene at the respective moments; and fitting the image of the bacterium obtained at the respective moments to an ellipsoidal shape, and setting the shape of the bacterium as the ellipsoidal shape and a position of the bacterium with respect to the solid surface.

### BRIEF DESCRIPTION OF THE DRAWINGS

The foregoing and other objects, features and advantages of the invention will become more apparent from the following more particular description of exemplary embodiments of the invention and the accompanying drawings. The drawings are not necessarily to scale, emphasis instead being placed upon illustrating the principles of the invention.

FIG. 1 illustrates a conventional method of setting the shape of a bacterium as spherical based on an image of the bacterium.

FIGS. 2 and 3 are flowcharts illustrating a method of tracking a swimming path of a bacterium according to an exemplary embodiment of the present invention.

FIG. 4 is a schematic diagram of an apparatus for obtaining an image of a bacterium from a solid surface.

FIG. 5 is an enlarged diagram of part “A” represented in FIG. 4.

FIGS. 6 to 9 are schematic diagrams illustrating a process of modeling the shape of a bacterium as an ellipsoid based on an image of the bacterium obtained by the apparatus shown in FIG. 4.

FIGS. 10 to 12 are graphs illustrating a comparison of swimming paths of a bacterium tracked by bacterial swimming path tracking methods according to an exemplary embodiment of the present invention and according to conventional art.

FIGS. 13 and 14 are diagrams illustrating distribution of centers of a bacterium in the cases of using the bacterial swimming path tracking methods according to an exemplary embodiment of the present invention and according to conventional art.

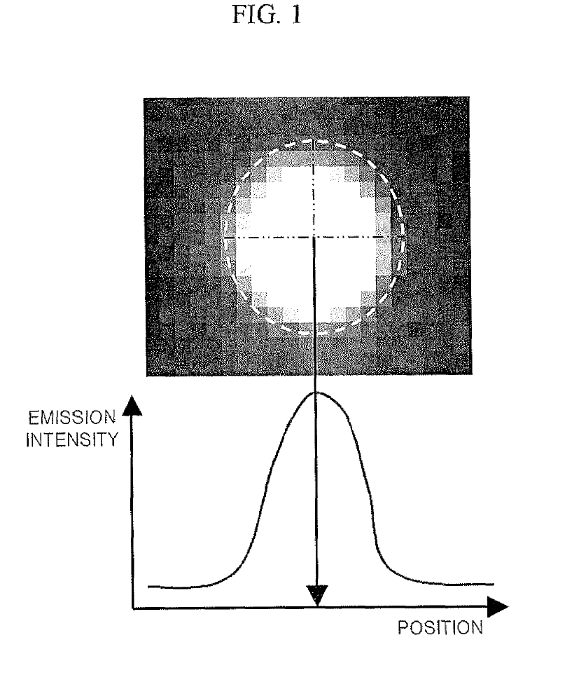

### DETAILED DESCRIPTION OF THE INVENTION

The present invention will now be described more fully hereinafter with reference to the accompanying drawings, in which exemplary embodiments of the invention are shown.

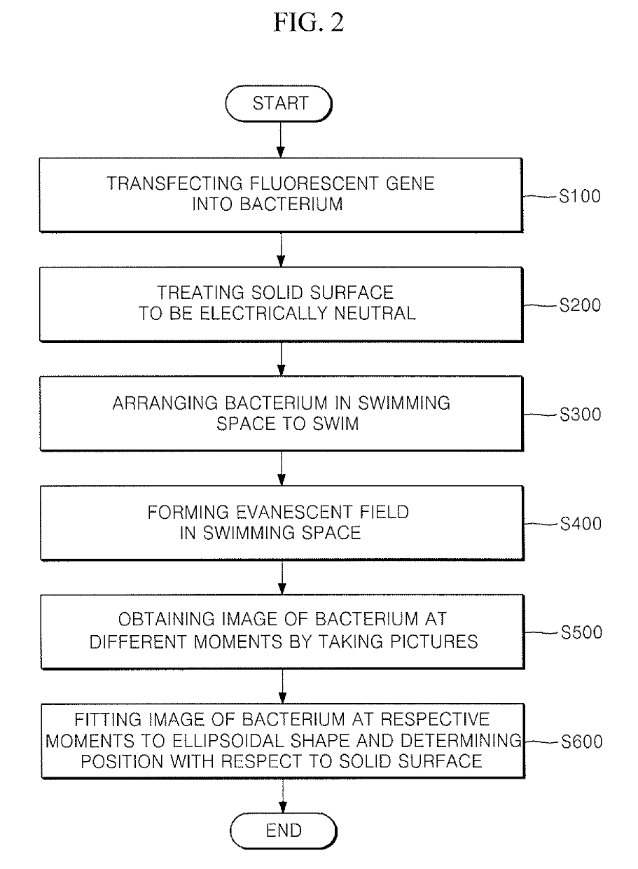

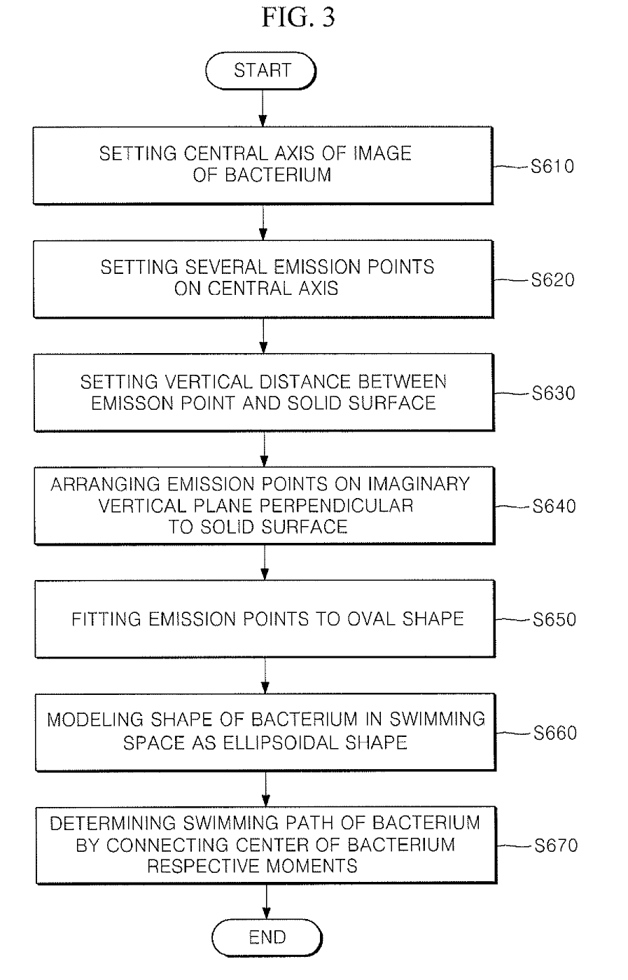

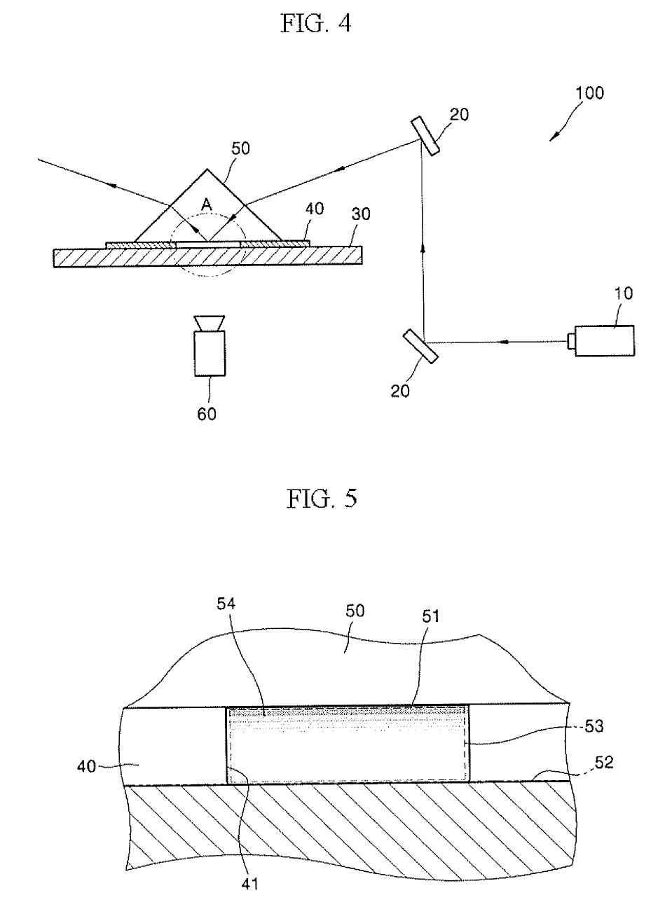

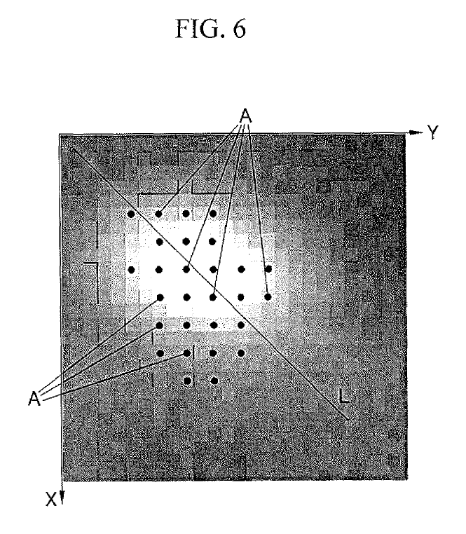

FIGS. 2 and 3 are flowcharts illustrating a method of tracking a swimming path of a bacterium according to an exemplary embodiment of the present invention, FIG. 4 is a schematic diagram of an apparatus for obtaining an image of a bacterium from a solid surface, FIG. 5 is an enlarged diagram of part “A” represented in FIG. 4, and FIGS. 6 to 9 are schematic diagrams illustrating a process of modeling the shape of a bacterium as an ellipsoid based on an image of the bacterium obtained by the apparatus shown in FIG. 4.

Referring to FIGS. 2 to 9, a swimming path of an ellipsoidal bacterium is tracked by the method of tracking a swimming path of a bacterium. In this embodiment, RP437 bacterium is used as the ellipsoidal bacterium. The RP437 bacterium is generally formed in an ellipsoidal shape that has a 2 μm major axis and an 800 nm minor axis. The method of tracking a swimming path of a bacterium, as illustrated in FIG. 2, comprises: a transfection step (S100); a solid surface treatment step (S200); a swimming step (S300); an evanescent field formation step (S400); an image acquisition step (S500); and a shape and position setting step (S600).

In the transfection step (S100), a fluorescent gene, for example, a known enhanced green fluorescent protein (eGFP) gene, is transfected into the RP 437 bacterium. Here, the eGFP gene is excited at an energy level with a wavelength of 488 nm. The transfection is a widely known process performed using a plasmid, and thus its detailed description will be omitted here. The RP437 bacterium is transfected with the eGFP genes to form RP437-pGFPmut2 bacterium. The RP437-pGFPmut2 bacterium is cultivated in a shaking incubator at 150 rpm for 6 hrs at 30° C.

In the solid surface treatment step (S200), a solid surface 50 illustrated in FIG. 4 is treated to be electrically neutral. This surface treatment is carried out by applying a surface treatment material produced by MERCK & Co., Inc. (Extran MA02) onto the solid surface, i.e., a bottom surface 51 thereof. The surface treatment cleans the bottom surface 51 of the solid and prevents electrostatic attraction between the bacterium and the bottom surface 51 of the solid. Here, the solid surface 50 is a prism formed of glass.

In the swimming step (S300), the RP437-pGFPmut2 bacterium is disposed in a swimming space 53 marked by a dotted line in FIG. 5 and swims therein. The swimming space 53 is formed between the bottom surface 51 of the solid and an imaginary surface 52 parallel to the bottom surface 51. Also, the swimming space 53 is formed by putting a cellophane tape 40 on a glass substrate 30, cutting a middle part of the cellophane tape 40 to form a concave well 41, and contacting the solid surface 50 onto the cellophane tape 40. Thus, as illustrated in FIG. 5, the swimming space 53 is formed in a space formed by an inner sidewall of the well 41 and the bottom surface 51 of the solid. Also, the swimming space 53 is fully filled with a medium. The swimming space 53 may be formed to various depths. However, to observe regular movement of the RP437-pGFPmut2 bacterium, the swimming space 53 may be formed to a depth of 10 μm or less.

In the evanescent field formation step (S400), an evanescent field 54 is formed to a certain thickness from the bottom surface 51 of the solid in the swimming space 53. The evanescent field 54, as illustrated in FIG. 4, is formed by reflecting a laser beam with a wavelength of 488 nm generated by an argon-ion laser generator 10 by a pair of reflectors 20, applying it to the solid surface 50, and totally reflecting it from the bottom surface 51 of the solid. It is known that the evanescent field 54 is an electromagnetic field whose intensity exponentially decreases in proportion to the distance from the surface of total reflection, i.e., the bottom surface 51 of the solid. Also, the thickness (Zₚ) of the evanescent field 54 may be theoretically calculated by the following <Formula 1>, as disclosed in Hecht E (2002), “Optics”, 4^{th} edition, Addison-Wesley, Reading, Mass., pp 124-127, and K. D. Kihm, A. Banerjee, C. K. Choi, T. Takagi, 2004, “Near-wall Hindered Brownian Diffusion of Nanoparticles Examined by Three-dimensional Ratiometric Total Internal Reflection Fluorescence Microscopy (3-D R-TIRFM)”, Experiments in Fluids, Vol. 37, pp 811-824.

$${z}_{p} = \frac{λ}{4 π {n}_{1} \sqrt{{sin}^{2} {θ}_{i} - {( \frac{{n}_{2}}{{n}_{1}} )}^{2}}} \qquad [ Formula 1 ]$$

Here, Z^{p} denotes the thickness of the evanescent field 54, θᵢ denotes the angle of incidence (rad) of a laser beam on the solid surface 50, n₁ denotes the refractive index of the solid surface 50, n₂ denotes the refractive index of a medium, and λ denotes the wavelength (nm) of a laser beam. With a θᵢ of 1.104 rad, an n₁ of 1.515 which is the refractive index of the solid formed of glass, an n₂ of 1.3338, and a λ of 488 nm, Formula 1 yields a Zₚ of about 170 nm. Accordingly, in this embodiment, the evanescent field is formed to a thickness of about 170 nm.

In the image acquisition step (S500), as illustrated in FIG. 4, a camera 60 is installed under the glass substrate 30 and pictures are taken of the RP437 bacterium in which the eGFP gene is expressed at different moments obtaining images of the bacterium at the respective moments. Here, the images of the RP437 bacterium in which the eGFP gene is expressed, that is, the RP437-pGFPmut2 bacterium, are images of a bacterium emitting light in the evanescent field. To be specific, when the entire bacterium is disposed in the evanescent field, an image of the entire bacterium may be obtained, but when only a part of the bacterium is disposed in the evanescent field, only a partial image of the bacterium can be obtained. In this embodiment, a partial image of the bacterium is obtained because the size of the bacterium is larger than the thickness of the evanescent field. Also, the image of the RP437-pGFPmut2 bacterium contains information on emission intensity. The picture of the bacterium is taken from the underside of the glass substrate and thus is a two-dimensional image on the bottom surface 51 of the solid as illustrated in FIG. 6. Here, the bottom surface 51 of the solid is set as an x-y plane for convenience.

In the image and position setting step (S600), the images of the RP437-pGFPmut2 bacterium obtained at the respective moments are fitted to an ellipsoidal shape, thereby setting the relative position of the RP437-pGFPmut2 bacterium with respect to the bottom surface 51 of the solid as well as the shape of the bacterium as an ellipsoid, which is the actual shape of the bacterium, unlike in the conventional art. More particularly, observing the process of fitting the bacterium to an ellipsoidal shape and setting its position relative to the bottom surface 51 of the solid with reference to FIG. 3, the shape and position setting step (S600) includes a central axis setting step (S610), an emission point setting step (S620), a vertical distance setting step (S630), an emission point arrangement step (S640), a fitting step (S650), a modeling step (S660), and a swimming path determination step (S670).

In the central axis setting step (S610), several emission points A are first set on the images of the RP437-pGFPmut2 bacterium obtained at the respective moments. Here, the emission points A are arranged at equal intervals as illustrated in FIG. 6 and have an emission intensity higher than a threshold. The threshold is set based on emission intensity of a noise part included in the image of the bacterium, which is set to be 30% higher than the emission intensity of the noise part in this embodiment. After that, a central axis L of the emission point A, as illustrated in FIG. 6, may be set on the bottom surface 51 of the solid, i.e., an x-y plane, by applying a known linear least square fitting method to the emission point A at the respective moments. As such, the central axis L at the respective moments corresponds to a central axis of the image of the RP437-pGFPmut2 bacterium at the moment.

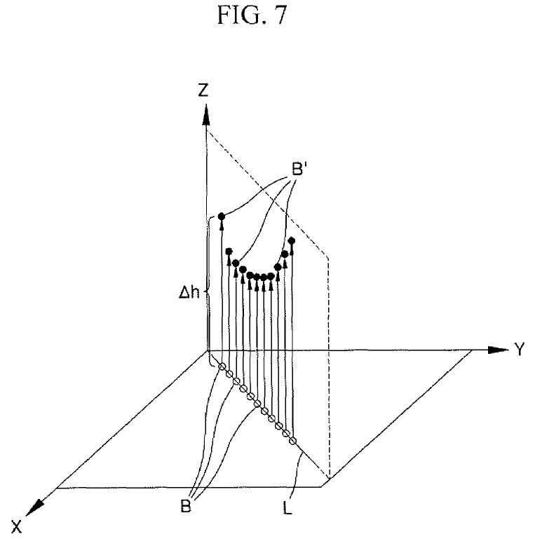

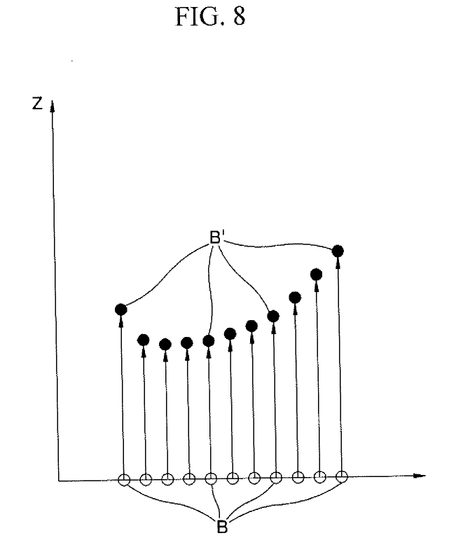

In the emission point setting step (S620), several emission points B are set on respective central axes L at the respective moments. Here, the emission points B are arranged at equal intervals as illustrated in FIGS. 7 and 8, and have an emission intensity higher than a threshold. And, the threshold is set to be 30% higher than the emission intensity of the noise part as described above.

In the vertical distance setting step (S630), the emission intensity of each emission point B arranged on the central axis at the respective moments is first compared with a predetermined reference value. Here, the reference value is emission intensity of the bacterium emitting at an interface between the bottom surface 51 of the solid and the evanescent field 54. After that, the vertical distance (Δh) between each emission point B and the bottom surface 51 of the solid is determined using the following <Formula 2>. Here, Formula 2 represents the relationship between energy level and displacement in the evanescent field, which is already disclosed in Hecht E (2002) “Optics”, 4^{th} edition, Addison-Wesley, Reading, Mass., pp 124-127, and K. D. Kihm, A. Banerjee, C. K. Choi, T. Takagi, 2004, “Near-wall Hindered Brownian Diffusion of Nanoparticles Examined by Three-dimensional Ratiometric Total Internal Reflection Fluorescence Microscopy (3-D R-TIRFM)”, Experiments in Fluids, Vol. 37, pp 811-824.

$$\frac{{I}_{1}}{{I}_{2}} = exp \vert - \frac{Δ h}{{z}_{p}} \vert \qquad [ Formula 2 ]$$

Here, I₁ denotes the emission intensity of each emission point B, I₂ denotes the reference value, Zₚ denotes the thickness (nm) of the evanescent field, and Δh denotes the vertical distance (nm) of each emission point B.

In the emission point arrangement step (S640), by using the vertical distance Δh of each emission point B set by Formula 2 at the respective moments, the emission point B is arranged on a Z-L plane as illustrated in FIG. 8. Here, the Z-L plane is an imaginary vertical plane perpendicular to the bottom surface 51 of the solid and including the central axis L.

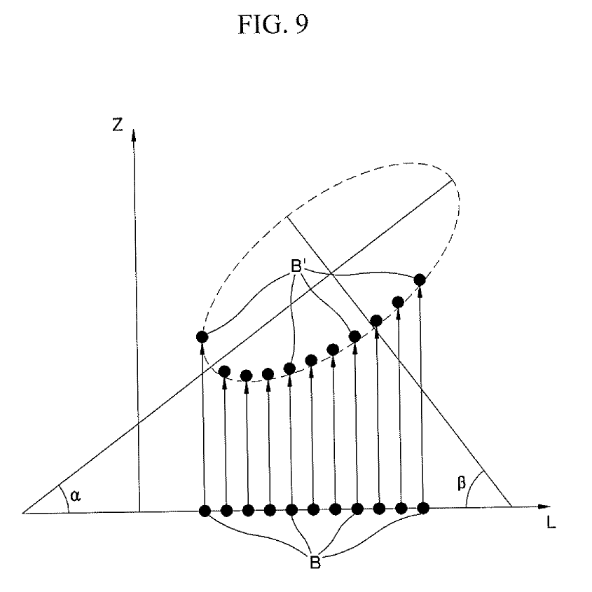

In the fitting step (S650), emission points B′ arranged on the imaginary vertical plane at the respective moments are fitted to an oval shape, thereby determining an image of the bacterium on the imaginary vertical plane as an oval, as illustrated in FIG. 9. Since the oval shape of the bacterium determined in this way may vary depending on various factors such as the sizes of major and minor axes, the sizes of at least the major and minor axes of the bacterium have to be determined before fitting it to the oval shape. For example, the RP437 bacterium is used in this embodiment, and thus the sizes of its major and minor axes are set to 2 μm and 800 nm, respectively, before fitting it to an oval shape.

In the modeling step (S660), by using the oval image of the bacterium determined in the fitting step (S650), the shape of the bacterium in the swimming space 53 at the respective moments is modeled as an ellipsoid. That is, the oval bacterium determined in the fitting step (S650) may be rotated based on the major axis, thereby modeling a three-dimensional image of the bacterium in the swimming space 53.

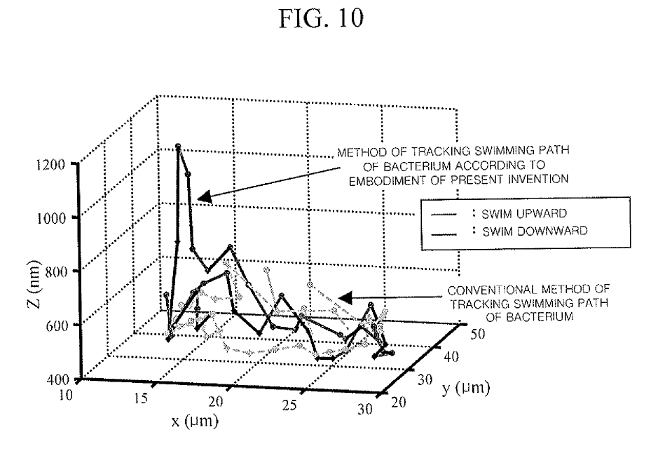

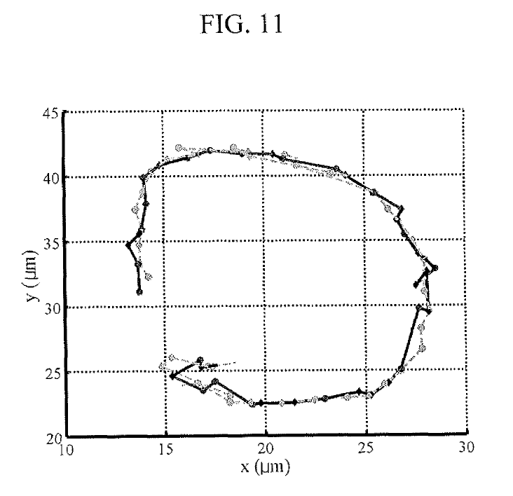

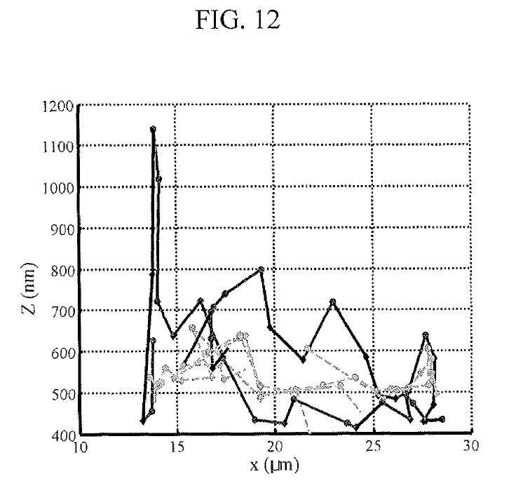

In the swimming path determination step (S670), centers of the bacterium at the respective moments are determined from the three-dimensional shape of the bacterium modeled in the modeling step (S660). Then, the centers of the bacterium at the respective moments are connected by straight line segments so that the swimming path of the bacterium in the swimming space may be obtained as illustrated in FIGS. 10 to 12. Also, positions of the bacterium at the respective moments, that is, an angle (α) between the major axis of the bacterium and the solid surface and/or an angle (β) between the minor axis of the bacterium and the solid surface, may be obtained.

FIGS. 10 to 12 illustrate the swimming path of the bacterium obtained by connecting the centers of the bacterium at the respective moments by straight line segments, which are determined as described above. FIGS. 10 to 12 also illustrate the swimming path of the bacterium obtained by a conventional method with a green dotted line. Moreover, the swimming path of the bacterium determined by the embodiment of the present invention is illustrated by red and blue straight lines. Here, the red line denotes the path of the bacterium swimming upward, and the blue line denotes the path of the bacterium swimming downward in the swimming space illustrated in FIG. 4.

As illustrated in FIGS. 10 to 12, the swimming paths of the bacterium on the x-y plane tracked by the method of the embodiment and the conventional method are similar. However; it can be noted that the swimming paths of the bacterium on the z-x plane obtained by the two methods are significantly different. That is, contrary to the conventional case, the bacterium fluctuates more intensively to move from the bottom surface of the solid. This is because the bacterium has an ellipsoidal shape. Apparently, when the bacterium is modeled as ellipsoidal in shape based on its emission image to more closely approximate its actual shape, there is much less error between the center of the modeled bacterium and the center of the actually swimming bacterium than in the conventional case.

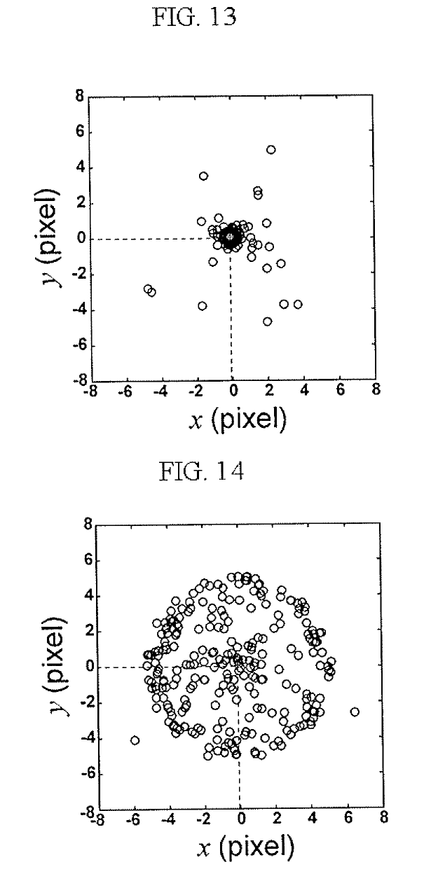

Meanwhile, in order to confirm that the error between the center of the modeled bacterium and the center of the actually swimming bacterium is much less than in the conventional case, an imaginary bacterium formed in an ellipsoidal shape (major axis: 2 μm, minor axis: 800 nm) was arranged in an imaginary evanescent field (thickness: 250 nm) and then randomly turned about its center. Thereby, an emitting part of the imaginary bacterium arranged in the evanescent field changed according to its state of rotation, thus changing the image of the imaginary bacterium. Accordingly, based on the different images of the imaginary bacterium obtained depending on the bacterium's rotation state, the center of the bacterium is set by the tracking method of the embodiment of the present invention, and FIG. 13 may be obtained by plotting the centers on the x-y plane. As such, by using the tracking method of the present embodiment, it can be confirmed that the center of the image set from the image of the imaginary bacterium corresponds closely to the center of the imaginary bacterium regardless of the rotation state of the imaginary bacterium illustrated in FIG. 13. Meanwhile, FIG. 14 illustrates a degree of dispersion of centers of the images of the imaginary bacterium set by the conventional tracking method with respect to the center of the imaginary bacterium. It can be confirmed that the center of the bacterium set by the conventional tracking method is quite different from the actual center. Units (pixels) of the x and y axes set in FIGS. 13 and 14 are pixels of the image of the imaginary bacterium, wherein 1 pixel represents 160 nm, and the center of the imaginary bacterium is set to (0, 0).

As described above, by using the bacterial swimming path tracking method according to the present embodiment, the bacterium's shape and the position relative to the bottom surface of a solid may be determined from an image of the bacterium including emission intensity while an ellipsoidal bacterium swims in a swimming space formed near the bottom surface of a solid. Particularly, in comparison with conventional methods, the ellipsoidal bacterium is modeled as ellipsoidal in shape to more closely approximate its actual shape, thereby more accurately obtaining the shape of the bacterium and the position relative to the solid surface, and therefore more accurately tracking the swimming path of the bacterium. Since the accurate tracking of the bacterium swimming path enables precise control of the movement of a bacterium, it contributes to effective application of a bacterium to industry, such as bio-filters, bio-pumps, bio-motors, and production of bio-energy.

According to the present invention with the aforementioned configuration, a bacterium may be modeled as ellipsoidal in shape based on an image of the bacterium obtained while the ellipsoidal bacterium swims near a solid surface, thereby exactly tracking the swimming path of the bacterium.

Exemplary embodiments of the present invention have been disclosed herein and, although specific terms are employed, they are used and are to be interpreted in a generic and descriptive sense only and not for purpose of limitation. Accordingly, it will be understood by those of ordinary skill in the art that various changes in form and details may be made without departing from the spirit and scope of the present invention as set forth in the following claims.

What is claimed is:

1. A method of tracking a swimming path of a bacterium which is formed in an ellipsoidal shape and swims in a swimming space formed between a solid surface and an imaginary surface parallel to the solid surface, the method comprising the steps of:
transfecting a fluorescent gene into the bacterium;
arranging the bacterium in the swimming space to swim;
totally reflecting light irradiated to the solid surface, and forming an evanescent field in the swimming space;
taking a picture of the bacterium expressing the fluorescent gene, which emits light in the evanescent field, at respective moments while the bacterium swims, and obtaining an image of the bacterium expressing the fluorescent gene at the respective moments; and
fitting the image of the bacterium obtained at the respective moments to an ellipsoidal shape, and setting the shape of the bacterium as the ellipsoidal shape and a position of the bacterium with respect to the solid surface.

2. The method according to claim 1, wherein the image of the bacterium comprises emission intensity as a two-dimensional image on the solid surface, and the step of setting the shape and the position comprises the steps of:
setting a central axis of the image of the bacterium from the bacterium images obtained at the respective moments;
setting several emission points each arranged on the central axis of the image of the bacterium set at the respective moments and having emission intensity;
comparing the emission intensity of each emission point at the respective moments with a predetermined reference value, and setting a vertical distance between each emission point and the solid surface;
arranging the emission point on an imaginary vertical plane which includes the central axis at the respective moments and is perpendicular to the solid surface using the vertical distance of the emission point; and
fitting the emission point arranged on the imaginary vertical plane at the respective moments to an oval shape, and determining the image of the bacterium on the imaginary vertical plane as an oval shape.

3. The method according to claim 2, further comprising the steps of:
modeling the shape of the bacterium in the swimming space at the respective moments as an ellipsoidal shape using the oval image of the bacterium determined in the fitting step; and
determining the centers of the bacterium at the respective moments from the shape of the bacterium modeled in the modeling step, and determining the swimming path of the bacterium by connecting the centers of the bacterium at the respective moments by straight line segments.

4. The method according to claim 2, wherein in the central axis setting step, after several emission points having an emission intensity higher than a threshold are arranged on the image of the bacterium, the central axes of the emission points are set by applying a linear least square fitting method to the emission points.

5. The method according to claim 2, wherein in the vertical distance setting step, the reference value is emission intensity of the bacterium at an interface between the solid surface and the evanescent field, and the vertical distance of the emission point is determined by the following formula:
$$\frac{{I}_{1}}{{I}_{2}} = exp \vert - \frac{Δ h}{{z}_{p}} \vert \qquad [ Formula ]$$
wherein, I₁ denotes the emission intensity of the emission point, I₂ denotes the reference value, Zₚ denotes the thickness of the evanescent field, and Δh denotes the vertical distance (nm) of the emission point.

6. The method according to any one of claims 1 to 5, further comprising the step of treating the solid surface to be electrically neutral.
